SEQUENCE LISTING (24 sequences)

SEQ ID NO: 1 - DNA, 20 bp, Synthetic construct
  source 1..20: mol_type = other DNA
ctgagcctgagctgcaattg

SEQ ID NO: 2 - DNA, 17 bp, Synthetic construct
  source 1..17: mol_type = other DNA
tgcccagcactcagcac

SEQ ID NO: 3 - DNA, 30 bp, Synthetic construct
  source 1..30: mol_type = other DNA
aggccacggacggctgtggtactctgctct

SEQ ID NO: 4 - DNA, 32 bp, Synthetic construct
  source 1..32: mol_type = other DNA
agccggttttccggctgagacgtccgtggcct

SEQ ID NO: 5 - DNA, 17 bp, Synthetic construct
  source 1..17: mol_type = other DNA
gctgagggtgcctggtg

SEQ ID NO: 6 - DNA, 21 bp, Synthetic construct
  source 1..21: mol_type = other DNA
cttttcaaggcacatgccaca

SEQ ID NO: 7 - DNA, 26 bp, Synthetic construct
  source 1..26: mol_type = other DNA
cgcgccgaggagcatccacctctgca

SEQ ID NO: 8 - DNA, 30 bp, Synthetic construct
  source 1..30: mol_type = other DNA
agccggttttccggctgagacctcggcgcg

SEQ ID NO: 9 - DNA, 18 bp, Synthetic construct
  source 1..18: mol_type = other DNA
ccctctgagcccctggtg

SEQ ID NO: 10 - DNA, 19 bp, Synthetic construct
  source 1..19: mol_type = other DNA
agcctgctgtgtttgcaca

SEQ ID NO: 11 - DNA, 27 bp, Synthetic construct
  source 1..27: mol_type = other DNA
acggacgcggagagaggcctgcactgg

SEQ ID NO: 12 - DNA, 32 bp, Synthetic construct
  source 1..32: mol_type = other DNA
agccggttttccggctgagactccgcgtccgt

SEQ ID NO: 13 - DNA, 17 bp, Synthetic construct
  source 1..17: mol_type = other DNA
cgagtccccgagcctga

SEQ ID NO: 14 - DNA, 19 bp, Synthetic construct
  source 1..19: mol_type = other DNA
gcacacgaatccggagcag

SEQ ID NO: 15 - DNA, 29 bp, Synthetic construct
  source 1..29: mol_type = other DNA
aggccacggacggagtaccgcagcgattg

SEQ ID NO: 16 - DNA, 32 bp, Synthetic construct; identical to SEQ ID NO: 4
  source 1..32: mol_type = other DNA

SEQ ID NO: 17 - DNA, 15 bp, Synthetic construct
  source 1..15: mol_type = other DNA
ccggcgcagagtccc

SEQ ID NO: 18 - DNA, 18 bp, Synthetic construct
  source 1..18: mol_type = other DNA
tgaggcttttcgaggcgc

SEQ ID NO: 19 - DNA, 24 bp, Synthetic construct
  source 1..24: mol_type = other DNA
cgcgccgaggcgcagaggcggacg

SEQ ID NO: 20 - DNA, 30 bp, Synthetic construct; identical to SEQ ID NO: 8
  source 1..30: mol_type = other DNA

SEQ ID NO: 21 - DNA, 18 bp, Synthetic construct
  source 1..18: mol_type = other DNA
ggcattcaaggagcggct

SEQ ID NO: 22 - DNA, 17 bp, Synthetic construct
  source 1..17: mol_type = other DNA
cctgctgtgtttgcgcg

SEQ ID NO: 23 - DNA, 26 bp, Synthetic construct
  source 1..26: mol_type = other DNA
acggacgcggagggaggcctgcgctg

SEQ ID NO: 24 - DNA, 32 bp, Synthetic construct; identical to SEQ ID NO: 12
  source 1..32: mol_type = other DNA

(19) United States
(12) Patent Application Publication
(10) Pub. No.: US 2025/0066838 A1
(43) Pub. Date: Feb. 27, 2025

(54) COMPOSITIONS AND METHODS FOR TET-ASSISTED OXIDATION OF METHYLATED NUCLEOTIDE BASES

(71) Applicant: Exact Sciences Innovations LTD., Oxford (GB)

(72) Inventors: Abram M. Vaccaro, Rio, WI (US); Brittany A. Otto, Madison, WI (US); Viatcheslav Katerov, Madison, WI (US); Hatim T. Allawi, Middleton, WI (US)

(21) Appl. No.: 18/848,614

(22) PCT Filed: Mar. 22, 2023
(86) PCT No.: PCT/US23/64810

§ 371 Date: Sep. 19, 2024

Related U.S. Application Data
(60) Provisional application No. 63/322,715, filed on Mar. 23, 2022.

(51) Int. Cl. C12Q 1/6806

(52) U.S. Cl. CPC C12Q 1/6806

(57) ABSTRACT

The present disclosure provides compositions and methods related to TET-assisted oxidation of methylated nucleotide bases. In particular, the present disclosure provides optimized oxidation methods which provide more efficient and complete oxidation. The disclosed methods may be used to identify and detect the methylated nucleotide bases, particularly in conjunction with known TET-assisted sequencing methods.

### CROSS REFERENCE TO RELATED APPLICATION

This application claims priority to U.S. provisional patent application Ser. No. 63/322,715 filed Mar. 23, 2022, which is incorporated herein by reference in its entirety.

### SEQUENCE LISTING

The text of the computer readable sequence listing filed herewith, titled “39895-601_SequenceListing”, created Mar. 22, 2023, having a file size of 22,256 bytes, is hereby incorporated by reference in its entirety.

### FIELD

The present disclosure provides compositions and methods related to TET-assisted oxidation of methylated nucleotide bases. In particular, the present disclosure provides optimized oxidation methods which provide more efficient and complete oxidation of methylated nucleotide bases in a target nucleic acid.

### BACKGROUND

Methylation, particularly at the 5 position of cytosine, is a major epigenetic modification in the mammalian genome that is associated with biological and pathological processes, including as well-accepted biomarkers of various diseases and cancer. In addition to methylation of cytosine residue, other modifications such as oxidation of methylated cytosine (5-mC) to 5-hydroxymethylcytosine (5-hmC), 5-formylcytosine (5-fC), 5-carboxylcytosine (5-caC), and methylation of adenine (A) to N⁶-methyladenine (6-mA), have also been identified as important epigenetic regulators. Therefore, detection of epigenetic modifications, such as methylation, has become critically important for scientific/diagnostic purposes.

Methylation is determined, for example, by a whole-genome, base-resolution, and quantitative sequencing method, such as bisulfite sequencing. However, bisulfite sequencing is damaging to the nucleic acid and expensive; therefore, current methylation sequencing is limited by being low-depth, targeted, or low-resolution and qualitative enrichment-based sequencing.

### SUMMARY

Embodiments of the present disclosure include methods for oxidizing a methylated nucleotide base (e.g., methylated cytosine (e.g., 5-methylcytosine (5mC) and 5-hydroxymethylcytosine (5hmC))).

The methods comprise providing a nucleic acid comprising or suspected of comprising at least one methylated nucleotide base and contacting the nucleic acid with an oxidation reaction mixture comprising a ten-eleven translocation (TET) family dioxygenase or an active fragment thereof, a TET family dioxygenase co-substrate and, a coordinated iron ion and/or a hydrogen peroxide reducing agent (e.g., a peroxidase (e.g., glutathione peroxidase)). In some embodiments, the iron ion is not coordinated with the TET family dioxygenase or the co-substrate.

Embodiments of the present disclosure also include methods for identifying or detecting a methylated nucleotide base in a target nucleic acid (e.g., DNA from a biological sample, genomic DNA, circulating free DNA, circulating tumor DNA, or any combination thereof). The methods comprise: providing the target nucleic acid; oxidizing the methylated nucleotide base using the methods disclosed herein; and detecting, directly or indirectly, the oxidized methylated nucleotide base.

In certain embodiments, a biological sample of the present disclosure comprises, but is not limited to, a sample comprising tissue, a cell, collection of cells, blood, plasma, serum, organ secretion, semen (seminal fluid), vaginal secretions, cerebral spinal fluid (CSF), saliva, mucus, urine, stool, sweat, pancreatic juice, gastric secretions, gastric fluid (gastric lavage), ascitic fluid, synovial fluid, pleural fluid (pleural lavage), pericardial fluid, peritoneal fluid, amniotic fluid, nasal fluid, optic fluid, breast milk, or any other bodily fluid comprising a desired nucleic acid, as well as cell culture supernatants. In some embodiments, the biological sample may include cells, secretions, or tissues from the lymph gland, breast, liver, lung, bile ducts, pancreas, mouth, stomach, colon, rectum, esophagus, small intestine, appendix, duodenum, polyps, gall bladder, anus, prostate, endometrium, vagina, ovary, cervix, skin, bladder, kidney, lung, and/or peritoneum. In other embodiments, the sample may contain diseased tissue or cells, or be suspected of containing diseased tissue or cells (e.g., a sample that may be cancerous, or may contain cancerous tissue or cells, or be suspected of being cancerous or suspected of containing cancerous tissue or cells). In some embodiments, the sample may be from a subject that has a disease or disorder (e.g., cancer), is suspected of having the disease or disorder, or is being screened to determine the presence of the disease or disorder.

In some embodiments, the methods further comprise contacting the target nucleic acid with a blocking group and a glucosyltranferase enzyme. In some embodiments, the methods further comprise converting the oxidized methylated nucleotide base to dihydroxyuracil via a reduction step (e.g., a borane reduction step).

In some embodiments, the detecting comprises sequencing the converted target nucleic acid. In some embodiments, the methods further comprise comparing the sequence of the converted target nucleic acid to a reference nucleic acid not comprising a methylated nucleotide base, and optionally, identifying at least one methylation biomarker in the target nucleic acid.

In some embodiments, the methods further comprise determining whether the at least one methylation biomarker is indicative of a disease or disorder (e.g., cancer, genomic imprinting disorder, immunodeficiency, autoimmune disease, metabolic disorders, psychological disorders, etc.), developmental state (e.g., embryonic development, differentiation status, aging status, etc.), or other nucleic acid structure or function (e.g., chromatin structure, genome stability, etc.).

Further embodiments of the present disclosure include systems or kits for oxidizing a methylated nucleotide comprising: a ten-eleven translocation (TET) family dioxygenase; a TET family dioxygenase co-substrate; and a coordinated iron ion and/or a hydrogen peroxide reducing agent (e.g., a peroxidase (e.g., glutathione peroxidase)).

In some embodiments, the TET family dioxygenase is selected from human TET1, TET2, and TET3; murine TET1, TET2, and TET3; Naegleria TET (NgTET); Coprinopsis cinerea (CcTET) and an active fragment, derivatives, or analogues thereof. In some embodiments, the TET family dioxygenase comprises TET1, TET2, TET3, CXXC4, an active fragment, derivatives, or analogues thereof.

In some embodiments, the co-substrate comprises oxoglutarate, or a derivative or analogue thereof. In some embodiments, the co-substrate comprises 2-oxoglutarate.

In some embodiments, the coordinated iron ion is provided as a hemoprotein or a fragment thereof. In some embodiments, the hemoprotein is catalase, or a fragment thereof. In some embodiments, the catalase, or fragment thereof is an enzymatically inactive catalase.

In some embodiments, the glutathione peroxidase is an enzymatically inactive glutathione peroxidase.

In some embodiments, the oxidation reaction mixture comprises an additional source of an iron ion (e.g., molecular iron). In some embodiments, the oxidation reaction mixture does not comprise an additional source of an iron ion (e.g., molecular iron).

In some embodiments, the oxidation reaction mixture comprises ascorbic acid. In some embodiments, the oxidation reaction mixture does not comprise ascorbic acid.

In some embodiments, the oxidation reaction mixture further comprises ethanol.

In some embodiments, the methods further comprise contacting the nucleic acid with a blocking group and/or a glucosyltranferase enzyme.

Other aspects and embodiments of the disclosure will be apparent in light of the following detailed description.

### BRIEF DESCRIPTION OF THE DRAWINGS

FIGS. 1A-1C and 1D-1F show the comparison of means for the percent conversion and percent recovery following exemplary oxidation reactions of methylated nucleotides in the presence of catalase, heat-killed catalase, and ethanol for three markers SDC2, QKI, and B3GALT6, respectively. Sample IDs are as follows: M-C, standard TET-assisted oxidation with catalase; M-C+EtOH, standard TET-assisted oxidation with catalase and ethanol; M-C-HK, standard TET-assisted oxidation with heat-killed catalase; M-C-HK+EtOH, standard TET-assisted oxidation with heat-killed catalase and ethanol; and M-stand, standard TET-assisted oxidation.

FIGS. 2A-2C and 2D-2F show the comparison of means for the percent conversion and percent recovery following exemplary oxidation reactions of methylated nucleotides in the presence of catalase and/or ethanol and the absence of molecular iron for three markers SDC2, QKI, and B3GALT6, respectively. Sample IDs are as follows: M-C, standard TET-assisted oxidation with catalase and molecular iron; M-C+EtOH, standard TET-assisted oxidation with catalase, molecular iron, and ethanol; M-C(no Fe), standard TET-assisted oxidation with catalase and without molecular iron; M-C+EtOH (no Fe), standard TET-assisted oxidation with catalase and ethanol and without molecular iron; M-stand, standard TET-assisted oxidation with molecular iron; and M-stand (No Fe), standard TET-assisted oxidation without molecular iron.

### DETAILED DESCRIPTION

Recently, TET-assisted sequencing methods that allow detection of methylated bases have been developed. Embodiments of the present disclosure include optimization of the TET enzyme oxidation resulting in increased oxidation percentages of the methylated bases. The increase in conversion efficiency facilitates more accurate identification and detection of methylated bases, and thereby more accurate diagnosis of diseases or disorders relating to differential methylation.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “methylation” refers to any and all processes by which methyl group(s) are added to a nucleic acid. For example, methylation may include, but is not limited to, the addition of methyl groups at positions C5 or N4 of cytosine or at the N6 position of adenine.

Accordingly, as used herein a “methylated nucleotide base” or “methylated nucleotide” refers to the presence of a methyl moiety on a nucleotide base, where the methyl moiety is not present in a recognized canonical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide.

As used herein, a “methylated nucleic acid” refers to a nucleic acid molecule that contains one or more methylated nucleotides. A nucleic acid molecule containing a methylated cytosine is considered methylated (e.g., the methylation state of the nucleic acid molecule is methylated). A nucleic acid molecule that does not contain any methylated nucleotides is considered unmethylated.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Methods for Oxidizing a Methylated Nucleotide Base

Embodiments of the present disclosure provide methods for oxidizing a methylated nucleotide base in a nucleic acid. The disclosed methods are suitable for use with any ten-eleven translocation (TET) family dioxygenase oxidation. The methods comprise providing a nucleic acid comprising or suspected of comprising at least one methylated nucleotide base and contacting the nucleic acid with an oxidation reaction mixture comprising a ten-eleven translocation (TET) family dioxygenase or an active fragment thereof, a TET family dioxygenase co-substrate and, a coordinated iron ion and/or a peroxidase (e.g., a glutathione peroxidase).

In some embodiments, the methylated nucleotide base comprises a methylated cytosine. In some embodiments, the methylated cytosine is selected from 5-methylcytosine (5mC) and 5-hydroxymethylcytosine (5hmC).

The methods comprise contacting the target nucleic acid with TET family dioxygenase or an active fragment, derivative, or analogue thereof. In some embodiments, the TET family dioxygenase comprises TET1, TET2, TET3, CXXC4, an active fragment, derivative, or analogue thereof, or any combination thereof. In some embodiments, the TET enzyme is selected from human TET1, TET2, and TET3; murine TET1, TET2, and TET3; Naegleria TET (NgTET); Coprinopsis cinerea (CcTET), an active fragment (e.g., the catalytic domain of mouse TET1 (mTET1CD)), derivatives, or analogues thereof.

In some embodiments, the TET family dioxygenase has at least 70%, at least 75%, at least 80%, at least 85%, at least 90%, at least 95%, at least 98%, at least 99%, or more sequence identity to any or all of the TET family dioxygenases listed above. In some embodiments, the TET family dioxygenase displays the characteristic enzymatic activity of the TET family of proteins.

TET proteins identified to date are large (˜180- to 230 -kDa) multidomain enzymes. Native TET proteins contain a conserved double-stranded β-helix (DSBH) domain, a cysteine-rich domain, and binding sites for Fe(II) and a co-substrate (e.g., 2-oxoglutarate (2-OG)) that together form the core catalytic region in the C terminus. The disclosed methods may also comprise a TET family dioxygenase co-substrate. In some embodiments, the co-substrate comprises oxoglutarate or a ketone derivative of glutaric acid. In some embodiments, the co-substrate comprises 2-oxoglutarate, also known as alpha-ketoglutarate.

The disclosed methods may further comprise a coordinated iron ion. The coordinated iron ion can comprise any array of bound molecules (e.g., amino acids, citric acid, vitamin C). The iron is typically Fe²⁺ or Fe³⁺.

In some embodiments, the coordinated iron ion is provided as a hemoprotein or a fragment thereof. Hemoproteins include, but are not limited to, hemoglobin, myoglobin, cytochromes, catalases, heme peroxidases and nitric oxide synthase.

In select embodiments, the hemoprotein is catalase, or a fragment thereof. In some embodiments, the catalase is an enzymatically inactive catalase, or fragment thereof. Catalase can be inactivated by known methods, including but not limited to, amino acid mutations, heat, addition of chloride, and singlet oxygen-mediated damage.

The disclosed method may additionally comprise addition of ascorbic acid to the reaction mixture comprising the TET family dioxygenase and nucleic acid. In some embodiments, the reaction mixture may comprise about 1 mM to about 100 mM ascorbic acid. In certain embodiments, the reaction mixture may comprise about 1 mM to about 50 mM ascorbic acid. In some embodiments, the reaction mixture comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 30 mM, about 40 mM, about 50 mM, about 60 mM, about 70 mM, about 80 mM, about 90 mM, or about 100 mM ascorbic acid.

In further embodiments, ascorbic acid may be omitted from the reaction mixture comprising the TET family dioxygenase and nucleic acid. In certain embodiments, when ascorbic acid is omitted from the reaction mixture, the reaction mixture comprises iron, for instance a coordinated iron ion.

The disclosed methods may further comprise a peroxidase, a fragment thereof, or other enzyme or polypeptide capable of catalyzing the breakdown of peroxides (e.g., a polypeptide having peroxidase activity). The term “peroxidase activity” is defined herein as an enzyme activity that converts a peroxide, e.g., hydrogen peroxide, to a less oxidative species, e.g., water. Exemplary peroxidases or peroxide-degrading enzymes include, but are not limited to, NADH peroxidase, NADPH peroxidase, fatty acid peroxidase, di-heme cytochrome c peroxidase, cytochrome c peroxidase, catalase, manganese catalase, invertebrate peroxinectin, eosinophil peroxidase, lactoperoxidase, myeloperoxidase, thyroid peroxidase, glutathione peroxidase, chloride peroxidase, ascorbate peroxidase, manganese peroxidase, lignin peroxidase, and cysteine peroxiredoxin. In some embodiments, the peroxidase is glutathione peroxidase.

The disclosed methods may further comprise adding ethanol to the reaction mixture comprising the TET family dioxygenase and the nucleic acid. The reaction mixture may comprise about 10 mM to about 100 mM ethanol. In some embodiments, the reaction mixture comprises about 10 mM, about 20 mM, about 30 mM, about 40 mM, about 50 mM, about 60 mM, about 70 mM, about 80 mM, about 90 mM, or about 100 mM ethanol.

The disclosed methods may further comprise adding a polynucleotide to the reaction mixture comprising the TET family dioxygenase and the nucleic acid. The polynucleotide may comprise DNA or RNA. The polynucleotide may be single stranded or double stranded. In some embodiments, the polynucleotide is Poly(A).

The method is not limited by the length of the polynucleotide. In some embodiments, the polynucleotide is between 100 and 500 nucleotides (e.g., about 100, about 150, about 200, about 250, about 300, about 350, about 400, about 450, or about 500 nucleotides) in length. In select embodiments, the polynucleotide is between 200 nucleotides and 300 nucleotides in length.

The method is not limited by the quantity of the polynucleotide added to the reaction mixture. In some embodiments, 100 ng-1,000 ng (e.g., about 100 ng, about 150 ng, about 200 ng, about 250 ng, about 300 ng, about 350 ng, about 400 ng, about 450 ng, about 500 ng, about 550 ng, about 600 ng, about 650 ng, about 700 ng, about 750 ng, about 800 ng, about 850 ng, about 900 ng, about 950 ng, or about 1,000 ng) is added to the reaction mixture.

### 3. Methods for Identifying or Detecting a Methylated Nucleotide Base

Embodiments of the present disclosure also provide methods for identifying or detecting one or more methylated nucleotide bases (e.g., 5-methylcytosine (5mC) and 5-hydroxymethylcytosine (5hmC)) in a target nucleic acid. The methods are suitable for use with TET-assisted sequencing methods and provide improvements for the oxidation of the methylated nucleotide base facilitating more accurate and complete detection of any or all methylated nucleotide bases in the target nucleic acid.

|0052| The target nucleic acid may include any nucleic acid comprising a methylated nucleotide base. In some embodiments, the target nucleic acid is DNA. The target DNA may include genomic DNA, circulating free DNA, circulating tumor DNA, or any combination thereof. In some embodiments, the methods are applied to a whole genome, and not limited to a specific region of the genome nucleic acid. Conversely, in some embodiments, the methods are limited to a specific region of a nucleic acid or nucleic acid sample (e.g., genomic DNA). In some embodiments, the target nucleic acid is RNA. In some embodiments, the nucleic acid comprises cytosine modifications (e.g., 5mC, 5hmC, 5-formylcytosine (5fC), and/or 5-carboxylcytosine (5caC)).

The nucleic acid can be a single nucleic acid molecule in a sample, or may be the entire population of nucleic acid molecules in a sample, or any portion thereof (e.g., whole genome or a subset thereof). The nucleic acid can be the native nucleic acid from the source (e.g., cells, tissue samples, etc.) or can be pre-converted into a high-throughput sequencing-ready form, for example by fragmentation, repair, and ligation with adaptors for sequencing. Thus, nucleic acids can comprise a plurality of nucleic acid sequences such that the methods described herein may be used to generate a library of target nucleic acid sequences that can be analyzed individually (e.g., by determining the sequence of individual targets) or in a group (e.g., by high-throughput or next generation sequencing methods).

The target nucleic acid can be obtained from an organism from the Monera (bacteria), Protista, Fungi, Plantae, and Animalia Kingdoms. The target nucleic acid may also be obtained from a virus. Nucleic acids may be obtained from a from a patient or subject, from an environmental sample, or from an organism of interest. In some embodiments, the target nucleic acid is obtained from a human subject/patient, including but not limited to, a human with a disease or disorder or a human suspected of having a disease or disorder (e.g., cancer). In some embodiments, the target nucleic acid is obtained from a biological sample from a human. For instance, from tissue, a cell, collection of cells, blood, plasma, serum, organ secretion, semen (seminal fluid), vaginal secretions, cerebral spinal fluid (CSF), saliva, mucus, urine, stool, sweat, pancreatic juice, gastric secretions, gastric fluid (gastric lavage), ascitic fluid, synovial fluid, pleural fluid (pleural lavage), pericardial fluid, peritoneal fluid, amniotic fluid, nasal fluid, optic fluid, breast milk, or any other bodily fluid comprising a desired nucleic acid, as well as cell culture supernatants. In some embodiments, the target nucleic acid may be obtained from cells, secretions, or tissues from the lymph gland, breast, liver, bile ducts, pancreas, mouth, stomach, colon, rectum, esophagus, small intestine, appendix, duodenum, polyps, gall bladder, anus, prostate, endometrium, vagina, ovary, cervix, skin, bladder, kidney, lung, and/or peritoneum. In other embodiments, the target nucleic acid may be obtained from a sample that is contains diseased tissue or cells, or is suspected of containing diseased tissue or cells (e.g., a sample that is cancerous, or contains cancerous tissue or cells, or is suspected of being cancerous or suspected of containing cancerous tissue or cells). In some embodiments, the target nucleic acid is obtained from a subject that has a disease or disorder (e.g., cancer), is suspected of having the disease or disorder, or is being screened to determine the presence of the disease or disorder.

The sample target nucleic acid for use in the methods of the present disclosure can be any quantity including, but not limited to, DNA from a single cell or bulk target nucleic acid samples. In some embodiments, the target nucleic acid sample comprises nanogram quantities of the target nucleic acid. In some embodiments, the target nucleic acid sample comprises microgram quantities of target nucleic acid. In some embodiments, the target nucleic acid sample comprises picogram quantities of target nucleic acid.

In some embodiments, the methods comprise contacting the target nucleic acid with TET family dioxygenase or an active fragment, derivative, or analogue thereof. In some embodiments, the TET family dioxygenase comprises TET1, TET2, TET3, CXXC4, an active fragment, derivative, or analogue thereof, or any combination thereof. In some embodiments, the TET enzyme is selected from human TET1, TET2, and TET3; murine TET1, TET2, and TET3; Naegleria TET (NgTET); Coprinopsis cinerea (CcTET), an active fragment (e.g., the catalytic domain of mouse TET1 (mTETICD)), derivatives, or analogues thereof.

In some embodiments, the TET family dioxygenase has at least 70%, at least 75%, at least 80%, at least 85%, at least 90%, at least 95%, at least 98%, at least 99%, or more sequence identity to any or all of the TET family dioxygenases listed above. In some embodiments, the TET family dioxygenase displays the characteristic activity of the TET family of proteins.

The disclosed methods may also comprise a TET family dioxygenase co-substrate. In some embodiments, the co-substrate comprises oxoglutarate or a ketone derivative of glutaric acid. In some embodiments, the co-substrate comprises 2-oxoglutarate, also known as alpha-ketoglutarate.

Descriptions of the coordinated iron ion provided elsewhere herein are applicable to the disclosed methods for identifying or detecting a methylated nucleotide base in a target nucleic acid.

The disclosed method may also comprise including ascorbic acid in the reaction mixture comprising the TET family dioxygenase and nucleic acid.

In further embodiments, ascorbic acid may be omitted from the reaction mixture comprising the TET family dioxygenase and nucleic acid. In certain embodiments, when ascorbic acid is omitted from the reaction mixture, the reaction mixture comprises iron, for instance a coordinated iron ion.

The disclosed methods may further comprise adding ethanol to the reaction mixture comprising the TET family dioxygenase and the nucleic acid.

The disclosed methods may also comprise adding a peroxidase, such as glutathione peroxidase, NADH peroxidase, NADPH peroxidase, fatty acid peroxidase, di-heme cytochrome c peroxidase, cytochrome c peroxidase, catalase, manganese catalase, invertebrate peroxinectin, eosinophil peroxidase, lactoperoxidase, myeloperoxidase, thyroid peroxidase, glutathione peroxidase, chloride peroxidase, ascorbate peroxidase, manganese peroxidase, lignin peroxidase, or cysteine peroxiredoxin to the reaction mixture comprising the TET family dioxygenase and the nucleic acid.

The disclosed methods may additionally comprise adding a polynucleotide, such as Poly(A) to the reaction mixture comprising the TET family dioxygenase and the nucleic acid.

In some embodiments of the invention, the methods further comprise amplifying the copy number of the modified target nucleic acid. In embodiments, this amplification step is performed prior to the step of detecting the sequence of the modified target nucleic acid.

The step of amplifying the copy number when the modified target nucleic acid is DNA may be accomplished by performing, for example, the polymerase chain reaction (PCR), primer extension, and/or cloning. The copy number of individual target DNAs can be amplified by PCR using primers specific for a particular target DNA sequence. Alternatively, a plurality of different modified target DNA sequences can be amplified by cloning into a DNA vector by standard techniques. In embodiments of the invention, the copy number of a plurality of different modified target DNA sequences is increased by PCR or other amplification technologies to generate a library for next generation sequencing where, e.g., double-stranded adapter DNA has been previously ligated to the sample DNA (or to the modified sample DNA) and amplification is performed using primers complimentary to the adapter DNA.

In some embodiments of the invention, the method comprises the step of detecting the oxidized methylated nucleotide base. The oxidized methylated nucleotide base may be detected by sequencing the modified target nucleic acid. The modified target nucleic acid contains dihydrouracil (DHU) at positions where one or more of 5mC, 5hmC, 5fC, and 5caC were present in the unmodified target nucleic acid. DHU acts as a T in DNA replication and sequencing methods. Thus, the cytosine modifications can be detected by any direct or indirect method that identifies a C to T transition known in the art. See for example, International PCT Appln. PCT/US2019/012627 and U.S. Pat. Nos. 10,533,213, 10,731,204, 10,767,216, and 11,072,818, each of which is incorporated herein by reference in its entirety. Such methods include sequencing methods such as Sanger sequencing, microarray, and next generation sequencing methods. The C to T transition can also be detected by restriction enzyme analysis where the C to T transition abolishes or introduces a restriction endonuclease recognition sequence.

In some embodiments of the invention, the detecting step comprises TET-assisted Pyridine Borane Sequencing (TAPS), a bisulfite-free DNA methylation sequencing method. As disclosed in International PCT Appln. PCT/US2019/012627, incorporated herein by reference in its entirety, TAPS comprises the use of mild enzymatic and chemical reactions to detect 5mC and 5hmC directly and quantitatively at base-resolution without affecting unmodified cytosines. In some embodiments, the methods of the present disclosure include are suitable for use with TAPS methods to detect 5mC and 5hmC. In some embodiments, the methods also detect 5-formylcytosine (5fC) and 5-carboxylcytosine (5caC) at base resolution without affecting unmodified cytosine.

In some embodiments, the methods further comprise providing a quantitative measure for frequency of the methylations in the target nucleic acid or at a specific location or region of the target nucleic acid. As such, in some embodiments, the present disclosure provides methods for identifying the location of one or more of methylated nucleotides in a target nucleic acid quantitatively with base-resolution.

In some embodiments, the methods of the present disclosure include identifying 5mC in target nucleic acid and providing a quantitative measure for the frequency of the 5mC modification at each location where the modification was identified in the DNA. In some embodiments, the percentages of the T at each transition location provide a quantitative level of 5mC at each location in the DNA. In accordance with these embodiments, methods for identifying 5mC can include the use of a blocking group. In other embodiments, methods for identifying 5mC do not require the use of a blocking group.

When a blocking group is used to identify 5mC without including 5hmC, the 5hmC in the sample is blocked so that it is not subject to conversion to 5caC and/or 5fC. In some embodiments, the 5hmC in the target nucleic acid are rendered non-reactive to the subsequent steps by adding a blocking group to the 5hmC. In one embodiment, the blocking group is a sugar, including a modified sugar, for example glucose or 6-azide-glucose (6-azido-6-deoxy-D-glucose). The sugar blocking group can be added to the hydroxymethyl group of 5hmC by contacting the DNA sample with uridine diphosphate (UDP)-sugar in the presence of one or more glucosyltransferase enzymes. In some embodiments, the glucosyltransferase is T4 bacteriophage β-glucosyltransferase (BGT), T4 bacteriophage α-glucosyltransferase (aGT), and derivatives and analogs thereof. BGT is an enzyme that catalyzes a chemical reaction in which a beta-D-glucosyl(glucose) residue is transferred from UDP-glucose to a 5-hydroxymethylcytosine residue in a nucleic acid.

In some embodiments, the methods of the present disclosure include identifying 5mC or 5hmC in a target nucleic acid. In some embodiments, the method provides a quantitative measure for the frequency the of 5mC or 5hmC modifications at each location where the modifications were identified in the target nucleic acid. In some embodiments, the percentages of the T at each transition location provide a quantitative level of 5mC or 5hmC at each location in the target nucleic acid. In accordance with these embodiments, the method for identifying 5mC or 5hmC provides the location of 5mC and 5hmC, but does not distinguish between the two cytosine modifications. Rather, both 5mC and 5hmC are converted to DHU. The presence of DHU can be detected directly, or the modified DNA can be replicated by known methods where the DHU is converted to T. In some embodiments, methods for identifying 5hmC include the use of a blocking group. In other embodiments, methods for identifying 5hmC do not require the use of a blocking group.

Because the 5mC and 5hmC are converted to 5fC and 5caC before conversion to DHU, any existing 5fC and 5caC in the DNA sample will be detected as 5mC and/or 5hmC. Given the extremely low levels of 5fC and 5caC in some nucleic acids, e.g., genomic DNA, under normal conditions, this will often be acceptable when analyzing methylation and hydroxymethylation in a target DNA sample. The 5fC and 5caC signals can be eliminated by protecting the 5fC and 5caC from conversion to DHU by, for example, hydroxylamine conjugation and EDC coupling, respectively.

The present disclosure also provides a method for identifying 5mC and identifying 5hmC in a DNA by performing the method for identifying 5mC on a first sample of the target nucleic acid, and performing the method for identifying 5mC or 5hmC on a second sample of the target nucleic acid. In some embodiments, the first and second samples are derived from the same sample. For example, the first and second samples may be separate aliquots taken from a sample comprising the target nucleic acid to be analyzed.

Because the 5mC and 5hmC (that is not blocked) are converted to 5fC and 5caC before conversion to DHU, any existing 5fC and 5caC in the DNA sample will be detected as 5mC and/or 5hmC. However, given the extremely low levels of 5fC and 5caC in genomic DNA under normal conditions, this will often be acceptable when analyzing methylation and hydroxymethylation in a DNA sample. The 5fC and 5caC signals can be eliminated by protecting the 5fC and 5caC from conversion to DHU by, for example, hydroxylamine conjugation and EDC coupling, respectively. In accordance with these embodiments, the method identifies the locations and percentages of 5hmC in the DNA through the comparison of 5mC locations and percentages with the locations and percentages of 5mC or 5hmC (together). Alternatively, the location and frequency of 5hmC modifications in a DNA can be measured directly. In some embodiments, identifying 5fC and/or 5caC provides the location of 5fC and/or 5caC, but does not distinguish between these two cytosine modifications. Rather, both 5fC and 5caC are converted to DHU, which is detected by the methods described herein.

Methods of the present disclosure can also include the step of converting the oxidized methylated nucleotide base (e.g., 5caC and/or 5fC) in the oxidized target nucleic acid to DHU. In some embodiments, this step comprises contacting the target nucleic acid sample with a reducing agent including, for example, a borane reducing agent such as pyridine borane, 2-picoline borane (pic-BH₃), borane, sodium borohydride, sodium cyanoborohydride, sodium triacetoxyborohydride, diborane, decaborane, borane tetrahydrofuran, borane-dimethyl sulfide, borane-N,N-diisopropylethylamine, borane-2-chloropyridine, borane-aniline, N,N-dimethylamine borane, tert-butylamine borane sodium triacetoxyborohydride, boron hydride, hydrazine or dibutylamine borane, morpholine borane, borane-ammonia complex (BH₃NH₃), dicyclohexylamine borane, morpholine borane, 4-methylmorpholine borane, alkali and tetramethylamine boranes (e.g. NaBH₄) and other-BH₃ containing complexes and/or derivatives. In some embodiments, the reducing agent is pyridine borane and/or pic-BH₃.

In some embodiments, the methods further comprise identifying at least one methylation biomarker and determining whether the methylation biomarker is indicative of a disease or disorder (e.g., cancer). In some embodiments, the methylation biomarker comprises a differentially methylated region (DMR). In some embodiments, the method further comprises classifying the sample based on the DMR as compared to a reference DMR. In some embodiments, the reference DMR corresponds to a non-diseased control or a disease or disorder control (e.g., non-cancerous control, or a cancerous control).

In some embodiments, and as described herein, the method further comprises identifying at least one methylation biomarker and determining a tissue-of-origin corresponding to the methylation biomarker. In some embodiments, the method further comprises classifying the sample based on the tissue-of-origin biomarker.

In some embodiments, the method further comprises identifying at least one sequence variant, and determining whether the sequence variant is indicative of a disease or disorder (e.g., cancer). For example, in some embodiments, the disclosed methods can also differentiate methylation from C-to-T genetic variants or single nucleotide polymorphisms (SNPs), and therefore, can be used to detect genetic variants. In some embodiments, methylations and C-to-T SNPs can result in different patterns. For example, methylations can result in T/G reads in an original top strand/original bottom strand, and A/C reads in strands complementary to these. In some embodiments, C-to-T SNPs can result in T/A reads in an original top strand/original bottom strand and strands complementary to these. This further increases the utility of the disclosed methods in providing both methylation information and genetic variants, and therefore mutations, in one experiment and sequencing run.

In accordance with the above embodiments, methods of the present disclosure include the use of the optimized TET oxidation and related sequencing analysis to generate information pertaining to any or all of: methylation signatures, methylation biomarkers, DNA fragment profiles, DNA sequence information (e.g., variants), and tissue-of-origin information in a single experiment to diagnose/detect a disease or disorder in a subject. The methods described herein can be used to diagnose/detect any disease or disorder associated with nucleic acid methylation.

In some embodiments, the methods described herein can be used to diagnose/detect any type of cancer. Types of cancers that can be detected/diagnosed using the methods of the present disclosure include, but are not limited to, lung cancer, melanoma, colon cancer, colorectal cancer, neuroblastoma, breast cancer, prostate cancer, renal cell cancer, transitional cell carcinoma, cholangiocarcinoma, brain cancer, non-small cell lung cancer, pancreatic cancer, liver cancer, gastric carcinoma, bladder cancer, esophageal cancer, mesothelioma, thyroid cancer, head and neck cancer, osteosarcoma, hepatocellular carcinoma, carcinoma of unknown primary, ovarian carcinoma, endometrial carcinoma, glioblastoma, Hodgkin lymphoma and non-Hodgkin lymphomas. In some embodiments, types of cancers or metastasizing forms of cancers that can be detected/diagnosed by the methods of the present disclosure include, but are not limited to, carcinoma, sarcoma, lymphoma, germ cell tumor and blastoma. In some embodiments, the cancer is invasive and/or metastatic cancer (e.g., stage II cancer, stage III cancer or stage IV cancer). In some embodiments, the cancer is an early stage cancer (e.g., stage 0 cancer, stage I cancer), and/or is not invasive and/or metastatic cancer.

In some embodiments, the methods of the present disclosure include treating a patient (e.g., a patient with cancer, with early-stage cancer, or who is suspected of having cancer). In some embodiments, the methods include determining a methylation biomarker as provided herein and administering a treatment to a patient based on the results of determining the methylation signature. The treatment can include administration of a pharmaceutical compound, a vaccine, performing a surgery, imaging the patient, and/or performing another test. In some embodiments, the methods of the present disclosure can be used as part of clinical screening, a method of prognosis assessment, a method of monitoring the results of therapy, a method to identify patients most likely to respond to a particular therapeutic treatment, a method of imaging a patient or subject, and a method for drug screening and development.

In some embodiments, methods of the present disclosure include diagnosing cancer in a subject. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example a methylation biomarker, which is indicative of the presence, severity, or absence of the condition (e.g., cancer).

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment of a subject based on one or more methylation biomarkers can be useful to separate subjects with good prognosis and/or low risk of developing cancer who will need no therapy or limited therapy from those more likely to develop cancer or suffer a recurrence of cancer who might benefit from more intensive treatments. As such, “making a diagnosis” or “diagnosing”, as used herein, is further inclusive of making a determination of a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the identification and assessment of one or more methylation biomarkers, as disclosed herein.

In some embodiments, methods of the present disclosure include determining whether to initiate or continue prophylaxis or treatment of a cancer in a subject. In some embodiments, the method comprises providing a series of biological samples over a time period from the subject; analyzing the series of biological samples to one or more methylation biomarkers as disclosed herein in each of the biological samples; and comparing any measurable change in the one or more methylation biomarkers in each of the biological samples. Any changes in the one or more methylation biomarkers over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation can be measured in each of the samples taken from different time points and qualitative and/or quantitative differences noted. A change in the one or more methylation biomarkers from the different samples can be correlated with risk for developing cancer, prognosis, determining treatment efficacy, and/or progression of the cancer in the subject. In some embodiments, the methods and compositions of the invention are for treatment or diagnosis of disease at an early stage, for example, before symptoms of the disease appear. In some embodiments, the methods and compositions of the invention are for treatment or diagnosis of disease at a clinical stage.

### 4. Systems or Kits

Embodiments of the present disclosure also provide systems or kits for oxidizing a methylated nucleotide (e.g., 5-methylcytosine (5mC) and 5-hydroxymethylcytosine (5hmC)). In some embodiments, the systems or kits comprise a TET family dioxygenase, a coordinated iron ion, and, optionally, a TET family dioxygenase co-substrate. Descriptions provide elsewhere for the TET family dioxygenase, coordinated iron ion, and, optionally, TET family dioxygenase co-substrate are applicable to the disclosed systems and kits.

In some embodiments, the TET family dioxygenase comprises TET1, TET2, TET3, CXXC4, an active fragment, derivative, or analogue thereof, or any combination thereof. In some embodiments, the TET enzyme is selected from human TET1, TET2, and TET3; murine TET1, TET2, and TET3; Naegleria TET (NgTET); Coprinopsis cinerea (CcTET), an active fragment (e.g., the catalytic domain of mouse TET1 (mTET1CD)), derivatives, or analogues thereof.

In some embodiments, the TET family dioxygenase has at least 70%, at least 75%, at least 80%, at least 85%, at least 90%, at least 95%, at least 98%, at least 99%, or more sequence identity to any or all of the TET family dioxygenases listed above. In some embodiments, the TET family dioxygenase displays the characteristic activity of the TET family of proteins.

The coordinated iron ion can comprise any array of bound molecules (e.g., amino acids, citric acid, vitamin C, transferrin, ferritin, and iron-cytochrome reductase). The iron is typically Fe²⁺ or Fe³⁺.

In some embodiments, the coordinated iron ion is bound by one or more porphyrins. For example, in some embodiments, the coordinated iron ion is provided as a heme coordination complex comprising an iron ion coordinated to a porphyrin acting as a tetradentate ligand, and, optionally, to one or two axial ligands. Any type of heme is suitable for use with the coordinated iron ion.

In some embodiments, the coordinated iron ion is provided as a hemoprotein or a fragment thereof. Hemoproteins include, but are not limited to, hemoglobin, myoglobin, cytochromes, catalases, heme peroxidases and nitric oxide synthase. In select embodiments, the hemoprotein is catalase, or a fragment thereof. In some embodiments, the catalase is an enzymatically inactive catalase, or fragment thereof. Catalase can be inactivated by known methods, including but not limited to, amino acid mutations, heat, addition of chloride, and singlet oxygen-mediated damage.

The systems or kits may also comprise a TET family dioxygenase co-substrate. In some embodiments, the co-substrate comprises oxoglutarate or a ketone derivative of glutaric acid. In some embodiments, the co-substrate comprises 2-oxoglutarate, also known as alpha-ketoglutarate.

The systems or kits may also comprise an ascorbic acid. In certain embodiments, when the systems or kits do not comprise ascorbic acid, the systems or kits comprise iron, for instance a coordinated iron ion. In another embodiment the systems or kits may further comprise ethanol.

The systems or kits may also comprise a peroxidase, such as glutathione peroxidase, NADH peroxidase, NADPH peroxidase, fatty acid peroxidase, di-heme cytochrome c peroxidase, cytochrome c peroxidase, catalase, manganese catalase, invertebrate peroxinectin, eosinophil peroxidase, lactoperoxidase, myeloperoxidase, thyroid peroxidase, glutathione peroxidase, chloride peroxidase, ascorbate peroxidase, manganese peroxidase, lignin peroxidase, or cysteine peroxiredoxin.

In other embodiments, the systems or kits may additionally comprise a polynucleotide, such as Poly(A).

The systems or kits may further comprise a borane reducing agent. In some embodiments, the borane reducing agent is selected from: pyridine borane, 2-picoline borane (pic-BH₃), borane, sodium borohydride, sodium cyanoborohydride, sodium triacetoxyborohydride, diborane, decaborane, borane tetrahydrofuran, borane-dimethyl sulfide, borane-N,N-diisopropylethylamine, borane-2-chloropyridine, borane-aniline, N,N-dimethylamine borane, tert-butylamine borane sodium triacetoxyborohydride, boron hydride, hydrazine or dibutylamine borane, morpholine borane, borane-ammonia complex (BH₃NH₃), dicyclohexylamine borane, morpholine borane, 4-methylmorpholine borane, alkali and tetramethylamine boranes (e.g. NaBH₄) and other-BH₃ containing complexes and/or derivatives. In some embodiments, the reducing agent is pyridine borane and/or pic-BH₃.

In some embodiments, the systems or kits further comprise a blocking group and/or a glucosyltransferase enzyme. In some embodiments, the blocking group is a sugar. In some embodiments, the sugar is a naturally-occurring sugar or a modified sugar, for example glucose or a modified glucose. In some embodiments, the blocking group functions with UDP linked to a sugar, for example UDP-glucose or UDP linked to a modified glucose in the presence of a glucosyltransferase enzyme, for example, T4 bacteriophage β-glucosyltransferase (BGT) and T4 bacteriophage α-glucosyltransferase (aGT) and derivatives and analogs thereof.

Such systems or kits may also be used for and comprise additional components necessary for the detection and identification of the methylated nucleotide. The systems or kits may further comprise sequencing reagents (e.g., primers, probes, nucleotides, buffers, control nucleic acid sequences, polymerases, etc.), restriction endonucleases, and the like for detecting the methylated nucleotide. The concepts, kits, and methods as described herein can be implemented on any system or instrument, including any manual, automated, or semi-automated system for sequencing reactions.

The systems or kits may include instructions for use in any of the methods described herein. Instructions included in the kit may be affixed to packaging material or may be included as a package insert. The instructions may be written or printed materials but are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), etc. As used herein, the term “instructions” may include the address of an internet site that provides the instructions.

The kits provided herein are in suitable packaging. Suitable packaging includes, but is not limited to, vials, bottles, jars, flexible packaging, and the like. Kits optionally may provide additional components such as buffers and interpretive information. Normally, the kit comprises a container and a label or package insert(s) on or associated with the container. In some embodiments, the disclosure provides articles of manufacture comprising contents of the kits described above.

### 5. EXAMPLES

The following are examples of the present invention and are not to be construed as limiting.

Preparation of catalase. Catalase was prepared as a 1 mg/mL solution and incubated for 30 min at 37° C. To completely denature catalase, the solution was incubated for 30 min at 70° C. Heat killed catalase showed equivalent activity to 0.08 U in each oxidation reaction compared to the average activity (4.85 U/oxidation reaction) of catalytically active catalase.

mTETI oxidation. In a control oxidation reaction up to 200 ng methylated DNA (Zymo, HCT-116 enzymatically methylated) was incubated in a 50 μl reaction containing 50 mM HEPES buffer (pH 8.0), 1 mM α-ketoglutarate, 2 mM ascorbic acid, 2.5 mM dithiothreitol, 100 mM NaCl, 100 μM ammonium iron (II) sulfate, 1.2 mM ATP and 4 μM mTET1 for 80 min at 37° C. For those reactions containing catalase, 2.5U of catalytically active or equivalent quantity of heat-killed catalase were incubated with the DNA, with or without the addition of 100 μM ammonium iron (II) sulfate, as indicated. In oxidation reactions containing ethanol, a 1M EtOH solution was added to the oxidation reactions at a final concentration of 20 mM.

After the oxidation reaction, 0.8 U of Proteinase K(New England Biolabs) was added to the reaction mixture and incubated for 1 h at 50° C. The product was cleaned up on Zymo-IC column.

Picoline borane reduction. Oxidized DNA was reduced in a 50 μl reaction containing 0.1M CHC Buffer pH 4 (Citric Acid, HEPES, CHE0.1S) and 0.1 M 2-picoline borane (Alfa Aesar) for 4 h at 37° C. and 850 r.p.m. in an Eppendorf ThermoMixer. The product was purified using Zymo-Spin columns.

Pre-Amplification. Following conversion, the DNA was purified using a standard silica column method (Zymo-IC spin columns) and processed through 2 cycles of linear pre-amplification using only reverse primers designed for each of three test markers (SDC2, QKI, and B3GALT6) of the DNA in both converted and non-converted samples. Exemplary pre-amplification parameters for processing the oxidized and reduced DNA are provided below. Each reaction comprised 0.075 μM of the reverse primers.

Pre-Amplification Cycling

| Stage | Temp/Time | # Cycles |
|---|---|---|
| Denaturation | 95° C./5′ | 1 |
| Amplification | 95° C./30″ | 2 |
| | 64° C./1′ | |
| Cooling | 4° C./Hold | 1 |

PCR/Strands per Reaction (LQAS). Following pre-amplification, the resulting sample, undiluted, was used in real-time PCR for each of three test markers. Exemplary PCR reaction cycling parameters are provided below. Each reaction comprised 0.2 μM of forward and reverse primers and 0.5 p M probes oligos.

LQAS Cycling

| Stage | Temp/Time | # Cycles | Acquisition |
|---|---|---|---|
| Denaturation | 95° C./3′ | 1 | None |
| Amplification | 95° C./20″ | 45 | None |
| | 63° C./1′ | | Single |
| | 70° C./30″ | | None |
| Cooling | 40° C./30″ | 1 | None |

Measuring Conversion Efficiencies. Assessment of the oxidation and overall conversion of the methylated cytosines the DNA sample can involve measuring the amount of DNA recovered after processing through the entire process and the amount of cytosines that remain unconverted from samples of the DNA before and after conversion. The presence and the slopes of the amplification curves were used as indicators of the conversion process and the efficiency of the conversion, respectively. LQAS assays specific for the converted DNA sequence and the unconverted DNA sequence were used to generate strands per reaction for each form and % conversion was determined using this equation->% Conversion=(LQAS TAPS Strands per reaction)/[(LQAS TAPS Strands per reaction)+ (LQAS WT Strands per reaction)]

### Oligo Sequences

TAPS Samples

| Marker | Oligo | Sequence |
|---|---|---|
| SDC2 | Forward | CTG AGC CTG AGC TGC AAT TG (SEQ ID NO: 1) |
| | Reverse | TGC CCA GCA CTC AGC AC (SEQ ID NO: 2) |
| | Probe | AGGCCACGGACG GCTGTGGTACTCTGCTCT/3C6/ (SEQ ID NO: 3) |
| | Arm 5 FAM hot | FAM.TCT[BHQ-1] |
| | | AGCCGGTTTTCCGGCTGAGACGTCCGTGGCCT.Spacer6 (SEQ ID NO: 4) |
| QKI | Forward | GCT GAG GGT GCC TGG TG (SEQ ID NO: 5) |
| | Reverse | CTT TTC AAG GCA CAT GCC ACA (SEQ ID NO: 6) |
| | Probe | CGC GCC GAG GAG CAT CCA CCT CTG CA/3C6/ (SEQ ID NO: 7) |
| | A1 HEX | HEX.TCT[BHQ-1] |
| | FRET_hot | AGCCGGTTTTCCGGCTGAGACCTCGGCGCG.Spacer6 (SEQ ID NO: 8) |
| B3GALT6 | Forward | CCCTCTGAGCCCCTGGTG (SEQ ID NO: 9) |
| | Reverse | AGCCTGCTGTGTTTGCACA (SEQ ID NO: 10) |
| | Probe | ACGGACGCGGAG AGAGGCCTGCACTGG/3C6/ (SEQ ID NO: 11) |
| | A3 Quasar670 | Quasar 670.TCT[BHQ-2] |
| | FRET_hot | AGCCGGTTTTCCGGCTGAGACTCCGCGTCCGT.Spacer 6 (SEQ ID NO: 12) |

WT Samples

| Marker | Oligo | Sequence |
|---|---|---|
| SDC2 | Forward | CGAGTCCCCGAGCCTGA (SEQ ID NO: 13) |
| | Reverse | GCACACGAATCCGGAGCAG (SEQ ID NO: 14) |
| | Probe | AGGCCACGGACG GAGTACCGCAGCGATTG/3C6/ (SEQ ID NO: 15) |
| | Arm 5 FAM hot | FAM.TCT[BHQ-1] |
| | | AGCCGGTTTTCCGGCTGAGACGTCCGTGGCCT.Spacer6 (SEQ ID NO: 16) |
| QKI | Forward | CCG GCG CAG AGT CCC (SEQ ID NO: 17) |
| | Reverse | TGA GGC TTT TCG AGG CGC (SEQ ID NO: 18) |
| | Probe | CGC GCC GAG GCG CAG AGG CGG ACG /3C6/ (SEQ ID NO: 19) |
| | A1 HEX | HEX.TCT[BHQ-1] |
| | FRET_hot | AGCCGGTTTTCCGGCTGAGACCTCGGCGCG.Spacer6 (SEQ ID NO: 20) |
| B3GALT6 | Forward | GGCATTCAAGGAGCGGCT (SEQ ID NO: 21) |
| | Reverse | CCTGCTGTGTTTGCGCG (SEQ ID NO: 22) |
| | Probe | ACGGACGCGGAG GGAGGCCTGCGCTG/3C6/ (SEQ ID NO: 23) |
| | A3 Quasar670 | Quasar 670.TCT[BHQ-2] |
| | FRET_hot | AGCCGGTTTTCCGGCTGAGACTCCGCGTCCGT.Spacer 6 (SEQ ID NO: 24) |

### Example 1

Catalase and Heat-Killed Catalase with and without Ethanol

200 ng of methylated DNA were oxidized using a standard TET oxidation reaction, or reactions including ˜2.5 units of catalase or an equivalent amount of heat-killed catalase in the presence or absence of ethanol, followed by picoline borane reduction as in standard TAP reactions and described above. Three different regions of the methylated DNA (SDC2, QKI, and B3GALT6) were amplified and analyzed for percent conversion and percent recovery.

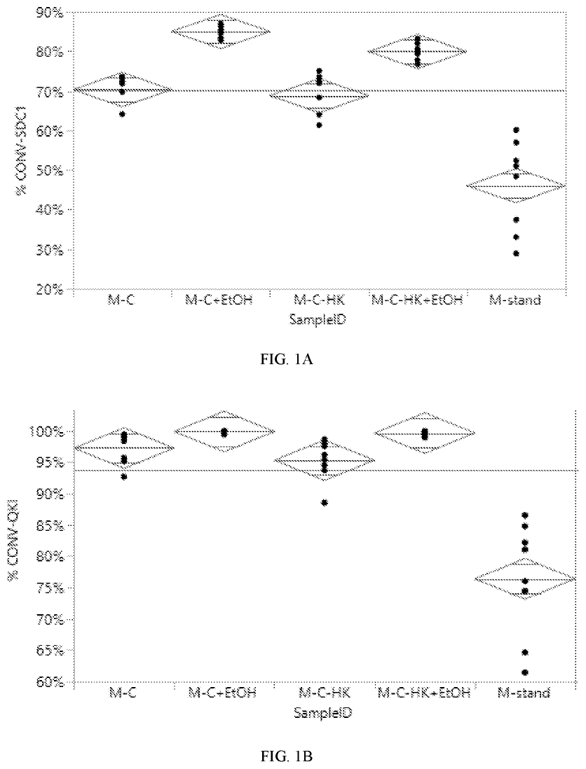

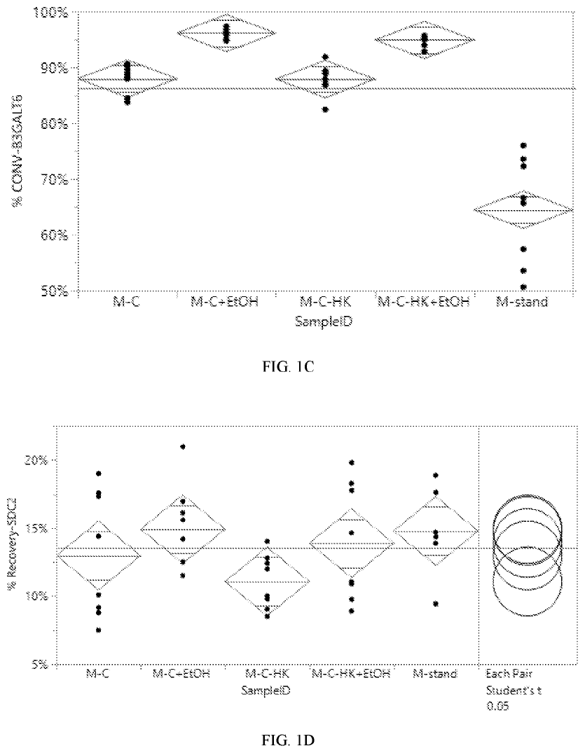

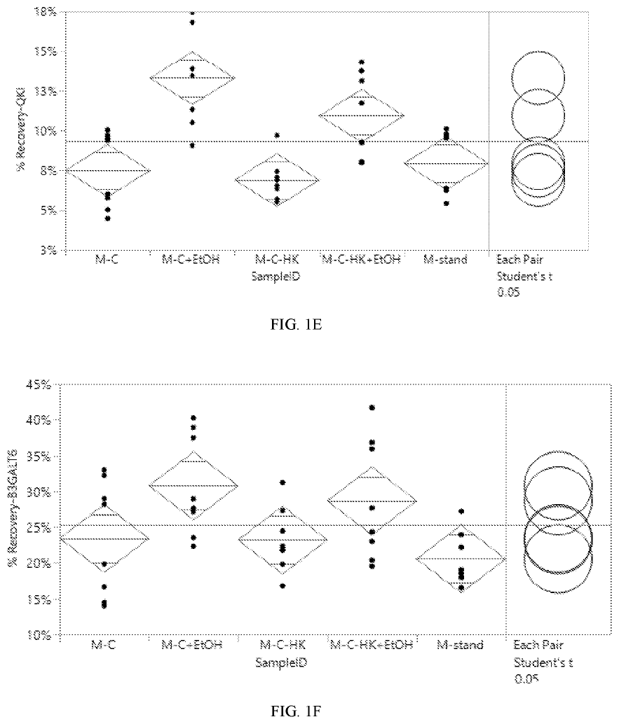

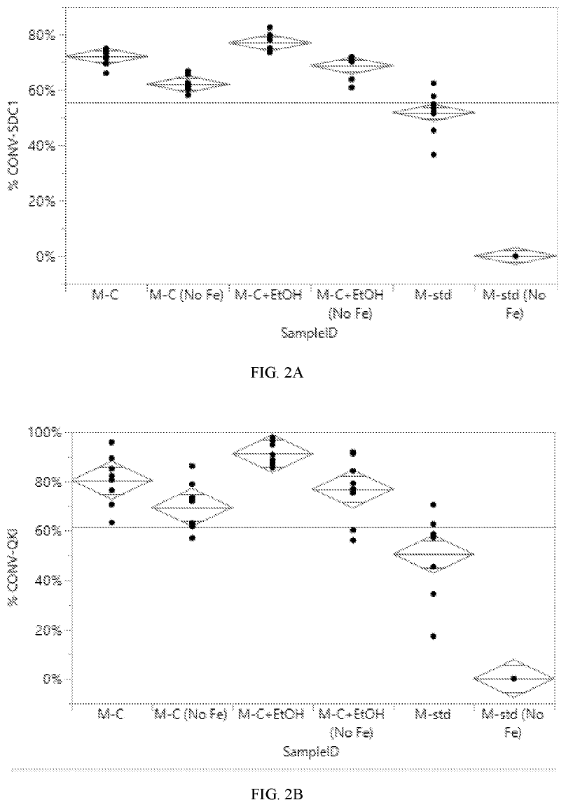

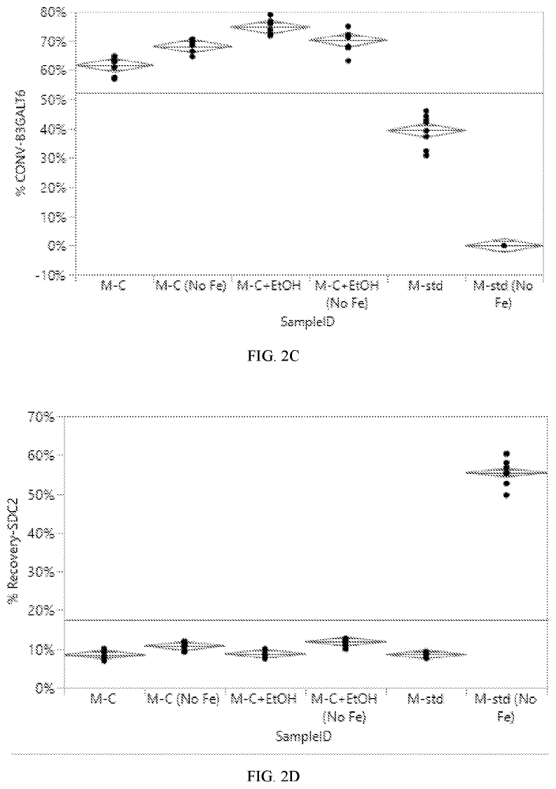

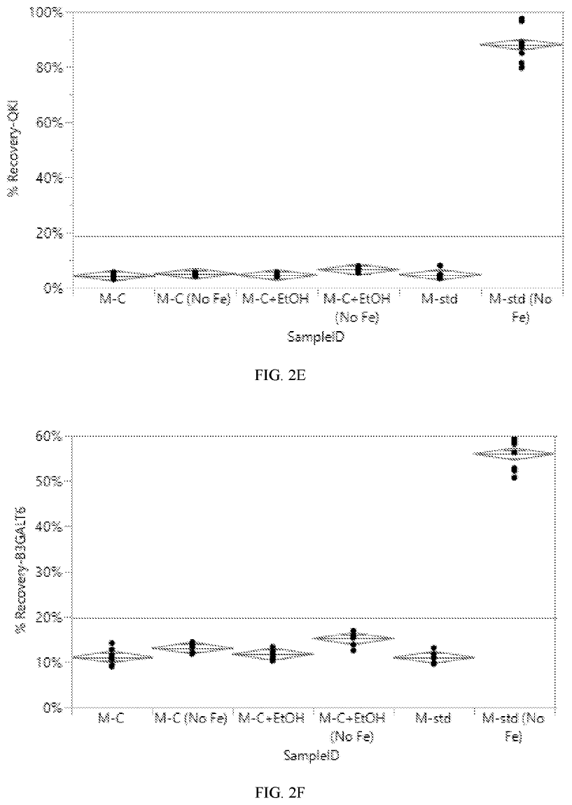

Surprisingly, heat-killed catalase increased the percent conversion of the oxidation reaction equal to active catalase (Tables 1-2). Thus, the data suggested that catalase is playing a role in the oxidation reaction unrelated or independent of its enzymatic activity. Interestingly, the addition of ethanol increased the percent conversion regardless of the enzymatic activity of the catalase. FIGS. 1A-1C and 1D-1F show the comparison of means for the percent conversion and percent recovery for SDC2, QKI, and B3GALT6, respectively.

TABLE 1
% Conversion

| SampleID | Catalase | EtOH | SDC2 | QKI | B3GALT6 |
|---|---|---|---|---|---|
| Univ. Methylated | 0 | 0 | 46% | 76% | 65% |
| DNA (HCT-116) | 2.5 U | 0 | 71% | 97% | 88% |
| | 2.5 U | 20 mM | 85% | 100% | 96% |
| | 2.5 U -Heat- | 0 | 69% | 96% | 88% |
| | Killed | | | | |
| | 2.5 U -Heat- | 20 mM | 80% | 100% | 95% |
| | Killed | | | | |

TABLE 2
% Conversion - Fold Diff from Standard

| SampleID | Catalase | EtOH | SDC2 | QKI | B3GALT6 |
|---|---|---|---|---|---|
| Univ. Methylated | 0 | 0 | — | — | — |
| DNA (HCT-116) | 2.5 U | 0 | 55% | 28% | 35% |
| | 2.5 U | 20 mM | 86% | 31% | 47% |
| | 2.5 U -Heat- | 0 | 50% | 26% | 35% |
| | Killed | | | | |
| | 2.5 U -Heat- | 20 mM | 74% | 31% | 46% |
| | Killed | | | | |

### Example 2

TAPS Oxidation without an Iron Source

To investigate if in the presence or absence of catalase, and optionally, ethanol, an additional iron source facilitates the oxidation reaction, similar experiments to those as in Example 1 were conducted with and without the inclusion of 100 μM ammonium iron (II) sulfate.

As shown in Tables 3-4, the inclusion of catalase in the oxidation reaction obviated the requirement for molecular iron necessary in the absence of catalase. As in Example 1, the inclusion of ethanol positively influenced the reaction.

TABLE 3

| | % Conversion | | | | | |
|---|---|---|---|---|---|---|
| SampleID | Catalase | EtOH | Fe | SDC2 | QKI | B3GALT6 |
| Univ. Methylated | 0 | 0 | Yes | 52% | 47% | 39% |
| DNA (HCT-116) | 0 | 0 | NO | 0% | 0% | 0% |
| | Catalase | 0 | Yes | 72% | 79% | 62% |
| | Catalase | 0 | NO | 62% | 69% | 68% |
| | Catalase | 20 mM | Yes | 77% | 91% | 75% |
| | Catalase | 20 mM | NO | 69% | 77% | 70% |

TABLE 4

| | % Conversion - Fold Diff from Standard | | | | | |
|---|---|---|---|---|---|---|
| SampleID | Catalase- | EtOH | Fe | SDC2 | QKI | B3GALT6 |
| Univ. | 0 | 0 | Yes | | | |
| Methylated | 0 | 0 | NO | −100% | −100% | −100% |
| DNA | Catalase | 0 | Yes | 40% | 67% | 58% |
| (HCT-116) | Catalase | 0 | NO | 20% | 46% | 74% |
| | Catalase | 20 mM | Yes | 50% | 93% | 91% |
| | Catalase | 20 mM | NO | 34% | 64% | 80% |

The strands per reaction are higher for both WT and TAPS DNA (Tables 5 and 6, respectively) when molecular iron was not present, indicating the molecular iron may be a causative agent in the DNA damage seen under standard reaction conditions. Thus, by removing the use of molecular iron more overall DNA is detected allowing for more accurate and reliable monitoring of methylation.

TABLE 5

| | | | WTSDC2 | | | WTQKI | | | WTB3GALT6 | | |
|---|---|---|---|---|---|---|---|---|---|---|---|
| Catalase | EtOH | Fe | strands | STDEV | % CV | strands | STDEV | % CV | strands | STDEV | % CV |
| 0 | 0 | Yes | 504 | 101 | 20% | 66 | 48 | 72% | 1,604 | 281 | 18% |
| 0 | 0 | NO | 6,700 | 391 | 6% | 2,359 | 172 | 7% | 13,358 | 842 | 6% |
| Catalase | 0 | Yes | 289 | 53 | 18% | 25 | 16 | 67% | 1,016 | 166 | 16% |
| Catalase | 0 | NO | 494 | 47 | 9% | 42 | 16 | 37% | 995 | 87 | 9% |
| Catalase | 20 mM | Yes | 242 | 22 | 9% | 11 | 6 | 57% | 712 | 97 | 14% |
| Catalase | 20 mM | NO | 447 | 30 | 7% | 40 | 21 | 52% | 1,068 | 51 | 5% |
| 0 | 0 | Yes | 12,074 | 320 | 3% | 2,679 | 119 | 4% | 23,875 | 782 | 3% |

TABLE 6

| | | | TAPSSDC2 | | | TAPSQKI | | | TAPSB3GALT6 | | |
|---|---|---|---|---|---|---|---|---|---|---|---|
| Catalase | EtOH | Fe | strands | STDEV | % CV | strands | STDEV | % CV | strands | STDEV | % CV |
| 0 | 0 | Yes | 538 | 78 | 15% | 59 | 19 | 32% | 1,029 | 124 | 12% |
| 0 | 0 | NO | 0 | 0 | 283% | 0 | 0 | — | 0 | 0 | — |
| Catalase | 0 | Yes | 748 | 123 | 16% | 91 | 8 | 9% | 1,638 | 273 | 17% |
| Catalase | 0 | NO | 810 | 92 | 11% | 94 | 11 | 11% | 2,131 | 172 | 8% |
| Catalase | 20 mM | Yes | 821 | 110 | 13% | 111 | 15 | 13% | 2,093 | 178 | 9% |
| Catalase | 20 mM | NO | 994 | 130 | 13% | 136 | 29 | 21% | 2,550 | 348 | 14% |

### Example 3

TAPS Oxidation with Nucleic Acids

To investigate the effectiveness of oxidation in the presence of various types of nucleic acids, standard TAPS oxidation reactions were conducted with and without the inclusion of 500 ng of PolyA (Millipore/Sigma Cat #10108626001), Fish DNA, or a tRNA (isolated from S. cerevisiae, Sigma-Aldrich).

As shown in Table 7, the inclusion of PolyA resulted in significantly higher conversion rates compared to the standard oxidation conditions. Furthermore, the addition of PolyA also resulted in significantly higher conversion rates when compared to the addition of either Fish DNA or tRNA.

TABLE 7
% Conversion

| SampleID | OX Condition | SDC2 | QKI | B3GALT6 |
|---|---|---|---|---|
| Univ. Methylated | Standard | 43.7% | 34.1% | 61.6% |
| DNA (HCT-116) | Standard + PolyA | 74.0% | 74.2% | 88.9% |
| | Standard + Fish DNA | 29.5% | 19.8% | 71.9% |
| | Standard + tRNA | 19.5% | 6.5% | 31.2% |

### Example 4

Oxidation with Glutathione Peroxidase

To investigate the effectiveness of oxidation in the presence of a peroxidase or an enzyme capable of breaking down peroxides, standard oxidation reactions, as described above, were conducted with glutathione peroxidase (GPx) or heat-killed glutathione peroxidase.

Glutathione peroxidase was able to improve the percent conversion of the mTET1 oxidation reaction to a level that is similar to the increase seen with catalase (Table 8). Heat-killed GPx, unlike heat-killed catalase, did not improve oxidation reaction (Table 9).

When the exogenous iron was excluded from the reactions, GPx was also able to allow mTETI to function (Table 10). This is particularly surprising because GPx is a selenoprotein which uses selenium to perform its peroxidase functions, and does not contain iron like catalase. However, heat-killed GPx improved percent conversion when exogenous iron was present (Table 11).

TABLE 8
% Conversion

| SampleID | OX Condition | SDC2 | QKI | B3GALT6 |
|---|---|---|---|---|
| Univ. Methylated | Standard | 66% | 68% | 80% |
| DNA (HCT-116) | Catalase (1.9 U) | 83% | 85% | 91% |
| | GPx (2.5 U) | 90% | 96% | 90% |
| | GPx (5 U) | 89% | 97% | 86% |
| | GPx (10 U) | 90% | 99% | 94% |

TABLE 9
% Conversion

| SampleID | OX Condition | SDC2 | QKI | B3GALT6 |
|---|---|---|---|---|
| Univ. Methylated | Standard | 42% | 56% | 66% |
| DNA (HCT-116) | Active GPx | 78% | 97% | 80% |
| | 70° C. Heat-killed | 57% | 60% | 68% |
| | GPx | | | |
| | 100° C. Heat-killed | 54% | 58% | 73% |
| | GPx | | | |

TABLE 10
% Conversion

| SampleID | Catalase | GPx | Fc | SDC2 | QKI | B3GALT6 |
|---|---|---|---|---|---|---|
| Univ. | 0 | 0 | Yes | 70% | 91% | 86% |
| Methylated | 0 | 0 | No | 0% | 0% | 0% |
| DNA | 665 ng | 0 | Yes | 88% | 95% | 97% |
| (HCT-116) | 665 ng | 0 | No | 83% | 94% | 97% |
| | 0 | 5 U | Yes | 91% | 96% | 91% |
| | 0 | 5 U | No | 82% | 100% | 80% |

TABLE 11
% Conversion

| | | Heat- | | | | |
|---|---|---|---|---|---|---|
| SampleID | GPx | Killed | Fe | SDC2 | QKI | B3GALT6 |
| Univ. | 0 | — | Yes | 80% | 98% | 92% |
| Methylated | 0 | — | No | 0% | 0% | 0% |
| DNA | 5 U | No | Yes | 95% | 100% | 96% |
| (HCT-116) | 5 U | No | No | 90% | 100% | 81% |
| | 5 U | Yes | Yes | 85% | 100% | 99% |
| | 5 U | Yes | No | 0% | 0% | 0% |

### Example 5

Oxidation with Catalase in the Absence of Ascorbic Acid and/or Iron

To investigate the effectiveness of oxidation in the presence of a catalase but it the absence of ascorbic acid, iron, or both ascorbic acid and iron, standard oxidation reactions, as described above, were conducted with catalytically active catalase.

The inclusion of catalase increased the effectiveness of reactions having iron, ascorbic acid, or both iron and ascorbic acid. However, the oxidation reaction is not effective in the absence of iron and ascorbic acid even in the presence of catalase (See Table 12). Thus, when catalase is included in the reaction you can remove iron or ascorbic acid but not both.

TABLE 12
% Conversion

| | | | | | Ascorbic | | | |
|---|---|---|---|---|---|---|---|---|
| SampleID | Catalase | | Fe | | Acid | SDC2 | QKI | B3GALT6 |
| Univ. | 0 | ng | 100 | μM | 2 mM | 56% | 66% | 86% |
| Methylated | 660 | ng | 100 | μM | 2 mM | 76% | 97% | 95% |
| DNA | 660 | ng | 100 | μM | 0 mM | 71% | 88% | 92% |
| (HCT-116) | 660 | ng | 0 | μM | 2 mM | 81% | 98% | 97% |
| | 660 | ng | 0 | μM | 0 mM | 0% | 0% | 0% |

### Example 6

Oxidation with Hydroquinone and Catalase

Standard oxidation reactions, as described above, were conducted with hydroquinone (HQ), at various concentrations, with or without catalase. Hydroquinone inhibits the oxidation reaction (Table 13) but catalase is able to, at least partially, rescue the oxidation (Table 14).

TABLE 13
% Conversion

| Sample ID | OX Condition | | SDC2 | QKI | B3GALT6 |
|---|---|---|---|---|---|
| Univ. Methylated | Standard | | 56% | 55% | 79% |
| DNA (HCT-116) | Catalase | | 71% | 86% | 89% |
| | 50 | μM HQ | 48% | 41% | 69% |
| | 100 | μM HQ | 31% | 10% | 46% |
| | 500 | μM HQ | 18% | 2% | 6% |
| | 1000 | μM HQ | 17% | 2% | 8% |

TABLE 14
% Conversion

| SampleID | OX Condition | SDC2 | QKI | B3GALT6 |
|---|---|---|---|---|
| Univ. Methylated | Standard | 52% | 68% | 62% |
| DNA (HCT-116) | Catalase | 74% | 97% | 92% |
| | 50 μM | 71% | 98% | 88% |
| | HQ + Catalase | | | |
| | 100 μM | 57% | 78% | 70% |
| | HQ + Catalase | | | |
| | 500 μM | 38% | 59% | 11% |
| | HQ + Catalase | | | |
| | 1000 μM | 29% | 24% | 1% |
| | HQ + Catalase | | | |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A method for oxidizing a methylated nucleotide base comprising:
contacting a nucleic acid comprising at least one methylated nucleotide base with an oxidation reaction mixture comprising:
a ten-eleven translocation (TET) family dioxygenase or an active fragment thereof,
a TET family dioxygenase co-substrate, and
a coordinated iron ion and/or a glutathione peroxidase,
wherein the iron ion is not coordinated with the TET family dioxygenase or the co-substrate.

2. The method of claim 1, wherein the TET family dioxygenase comprises human TET1, human TET2, human TET3, murine TET1, murine TET2, murine TET3, Naegleria TET (NgTET), Coprinopsis cinerea (CcTET), an active fragment, derivative, or analogue thereof.

3. The method of claim 1, wherein the TET family dioxygenase comprises TET1, TET2, TET3, CXXC4, an active fragment, derivative, or analogue thereof.

4. The method of any of claim 1-3, wherein the co-substrate comprises oxoglutarate, or a derivative or analogue thereof.

5. The method of any of claims 1-4, wherein the co-substrate comprises 2-oxoglutarate.

6. The method of any of claims 1-5, wherein the coordinated iron ion is provided as a hemoprotein or a fragment thereof.

7. The method of claim 6, wherein the hemoprotein is catalase.

8. The method of claim 7, wherein the catalase is an enzymatically inactive catalase.

9. The method of any of claims 1-8, wherein the glutathione peroxidase is an enzymatically inactive glutathione peroxidase.

10. The method of any of claims 1-9, wherein the oxidation reaction mixture comprises an additional source of an iron ion.

11. The method of claim 10, wherein the oxidation reaction mixture does not comprise ascorbic acid.

12. The method of any of claims 1-9, wherein the oxidation reaction mixture does not comprise an additional source of an iron ion.

13. The method of claim 12, wherein the oxidation reaction mixture comprises ascorbic acid.

14. The method of any of claims 1-13, wherein the oxidation reaction mixture further comprises ethanol.

15. The method of any of claims 1-14, wherein the methylated nucleotide base is a methylated cytosine.

16. The method of claim 15, wherein the methylated cytosine is selected from 5-methylcytosine (5mC) and 5-hydroxymethylcytosine (5hmC).

17. The method of any of claims 1-16, further comprising contacting the nucleic acid with a blocking group and a glucosyltranferase enzyme.

18. A method for identifying or detecting a methylated nucleotide base in a target nucleic acid comprising:
a) oxidizing the methylated nucleotide base comprising the steps of:
contacting a nucleic acid comprising at least one methylated nucleotide base with an oxidation reaction mixture comprising:
a ten-eleven translocation (TET) family dioxygenase or an active fragment thereof,
a TET family dioxygenase co-substrate, and
a coordinated iron ion and/or a glutathione peroxidase, wherein the iron ion is not coordinated with the TET family dioxygenase or the co-substrate; and
c) detecting the oxidized methylated nucleotide base.

19. The method of claim 18, wherein the TET family dioxygenase comprises human TET1, human TET2, human TET3, murine TET1, murine TET2, murine TET3, Naegleria TET (NgTET), Coprinopsis cinerea (CcTET), an active fragment, derivative, or analogue thereof.

20. The method of claim 19, wherein the TET family dioxygenase comprises TET1, TET2, TET3, CXXC4, an active fragment, derivatives, or analogues thereof.

21. The method of any of claim 18-20, wherein the co-substrate comprises oxoglutarate, or a derivative or analogue thereof.

22. The method of any of claims 18-21, wherein the co-substrate comprises 2-oxoglutarate.

23. The method of any of claims 18-22, wherein the coordinated iron ion is provided as a hemoprotein or a fragment thereof.

24. The method of claim 23, wherein the hemoprotein is catalase.

25. The method of claim 24, wherein the catalase is an enzymatically inactive catalase.

26. The method of any of claims 18-25, wherein the glutathione peroxidase is an enzymatically inactive glutathione peroxidase.

27. The method of any of claims 18-26, wherein the oxidation reaction mixture comprises an additional source of an iron ion.

28. The method of claim 18-27, wherein the oxidation reaction mixture does not comprise ascorbic acid.

29. The method of any of claims 18-26, wherein the oxidation reaction mixture does not comprise an additional source of an iron ion.

30. The method of claim 29, wherein the oxidation reaction mixture comprises ascorbic acid.

31. The method of any of claims 18-20, wherein the oxidation reaction mixture further comprises ethanol.

32. The method of any of claims 18-31, wherein the methylated nucleotide base is a methylated cytosine.

33. The method of claim 32, wherein the methylated cytosine is selected from 5-methylcytosine (5mC) and 5-hydroxymethylcytosine (5hmC).

34. The method of any of claims 18-33, further comprising contacting the target nucleic acid with a blocking group and a glucosyltranferase enzyme.

35. The method of any of claims 18-34, further comprising converting the oxidized methylated nucleotide base to dihydroxyuracil with reduction.

36. The method of any of claims 18-35, wherein the detecting comprises sequencing the converted target nucleic acid.

37. The method of claim 36, further comprising comparing the sequence of the converted target nucleic acid to a reference nucleic acid not comprising a methylated nucleotide base, and optionally, identifying at least one methylation biomarker in the target nucleic acid.

38. The method of claim 37, further comprising determining whether the at least one methylation biomarker is indicative of a disease or disorder.

39. The method of claim 38, wherein the disease or disorder comprises cancer.

40. The method of any of claims 18-39, wherein the target nucleic acid comprises genomic DNA, circulating free DNA, circulating tumor DNA, or any combination thereof.

41. A system or kit for oxidizing a methylated nucleotide comprising:
a ten-eleven translocation (TET) family dioxygenase;
a TET family dioxygenase co-substrate, and
a coordinated iron ion and/or a glutathione peroxidase, wherein the iron ion is not coordinated with the TET family dioxygenase or the co-substrate.

42. The system or kit of claim 41, wherein the TET family dioxygenase comprises human TET1, human TET2, human TET3, murine TET1, murine TET2, murine TET3, Naegleria TET (NgTET), Coprinopsis cinerea (CcTET), an active fragment, derivative, or analogue thereof.

43. The system or kit of claim 42, wherein the TET family dioxygenase comprises TET1, TET2, TET3, CXXC4, an active fragment, derivatives, or analogues thereof.

44. The system or kit of any of claim 41-43, wherein the co-substrate comprises oxoglutarate, or a derivative or analogue thereof.

45. The system or kit of any of claims 41-44, wherein the co-substrate comprises 2-oxoglutarate.

46. The system or kit of any of claims 41-45, wherein the coordinated iron ion is a hemoprotein or a fragment thereof.

47. The system or kit of claim 46, wherein the hemoprotein is catalase.

48. The system or kit of claim 47, wherein the catalase is an enzymatically inactive catalase.

49. The system or kit of any of claims 41-48, wherein the glutathione peroxidase is an enzymatically inactive glutathione peroxidase.

50. The system or kit of any of claims 41-49, further comprising an additional source of an iron ion.

51. The system or kit of any of claims 41-50, wherein the system or kit further comprises ethanol.

52. The system or kit of any of claims 41-51, wherein the system or kit further comprises ascorbic acid.

53. The system or kit of any of claims 41-52, wherein the system or kit further comprises a blocking group and/or a glucosyltransferase enzyme.

54. Use of a kit of any of claims 41-53 for oxidizing a methylated nucleotide base.

55. The use of claim 54, wherein the methylated nucleotide base is a methylated cytosine.

56. The use of claim 55, wherein the methylated cytosine is selected from 5-methylcytosine (5mC) and 5-hydroxymethylcytosine (5hmC).
